(19) United States
(12) Patent Application Publication
(10) Pub. No.: US 2014/0360522 A1
(43) Pub. Date: Dec. 11, 2014

(54) HAIR MANAGEMENT PROCESS

(71) Applicant: Raymond Oliver Anderson, Bridgeport, CT (US)

(72) Inventor: Raymond Oliver Anderson, Bridgeport, CT (US)

(21) Appl. No.: 13/910,559

(22) Filed: Jun. 5, 2013

(51) Int. Cl. A45D 7/04; A45D 7/06; A61K 8/92

(52) U.S. Cl. CPC A45D 7/04; A61K 8/92; A45D 7/06
USPC 132/206; 132/203; 132/207; 132/202

(57) ABSTRACT

A hair management process includes washing the hair and scalp, towel drying the hair and leaving the hair damp, applying an oil composition to the scalp, applying hair moisturizer to the hair and scalp, covering the hair with a cap, removing the cap and applying hair moisturizer to the hair, applying hair mousse on top of the moisturizer and styling the hair.

### CROSS-REFERENCE TO RELATED APPLICATIONS

This application claims the benefit of U.S. Provisional Patent Application No. 61/653,425 filed on Jun. 5, 2012, which is incorporated herein by reference.

### TECHNICAL FIELD OF THE INVENTION

The present invention relates to a cosmetic hair care product and more particularly to process for applying a cosmetic hair product.

### BACKGROUND OF THE INVENTION

In hair styling, there are presently many techniques which can damage hair. For example, hair can be damaged from hair dryers, curling or hot irons, or over exposure to heat. In fact, scorching heat can permanently damage hair. Hair bleaching which lifts the hairs natural color tends to dry the hair out at the follicle. A hair relaxer is a chemical processes which strips hair of it natural nutrients and oils. Over the counter hair products that contain petroleum or petrolatum suffocates the hair, weighing it down and taking away bounce ability to have the hair behave as desired. Shampoo hair products which contain sodium lauryl sulfate strip the hair of natural oils, leaving the hair dry and unmanageable. Hair which has not been washed enough builds up dirt inhibiting growth. Hair styles which damage hair such as hair braiding, using tight rollers, hair ties and hair clips, cause a slow sparse hair lines and can lead to permanent hair loss. Hair which is over processed with relaxers after dyeing the hair or dyeing already relaxed hair has led to hair loss. Another source of hair damage is from detangling the hair. Split ends which travel up the hair shaft and cause more damage to the hair.

### SUMMARY OF THE INVENTION

According to an embodiment of the present invention, a hair management process comprises the steps of: washing the hair and scalp; towel drying the hair and leaving the hair damp; applying an oil composition to the scalp; applying hair moisturizer to the hair and scalp; covering the hair with a cap; removing the cap and applying hair moisturizer to the hair; applying hair mousse on top of the moisturizer; and styling the hair.

According to an embodiment of the present invention, a hair management process for kinky, curly hair comprises the steps of: shampooing and conditioning the hair in a natural state; drying the hair and leaving the hair damp; adding oil and moisturizing solution to the hair and scalp; covering the hair a hair cap; removing the hair cap after about 6 to about 8 hours; moisturizing the hair and adding hair mousse to the top of the hair; and styling the hair.

According to another embodiment of the present invention, a hair management process for chemically relaxed curly hair comprises the steps of: shampooing and conditioning the hair; drying the hair and leaving the hair damp; adding oil and moisturizing solution to the hair and scalp; covering the hair a hair cap; removing the hair cap after about 6 to about 8 hours; moisturizing the hair and adding hair mousse to the top of the hair; drying the hair; repeating steps daily except shampooing and conditioning; and rinsing hair daily. According to yet another embodiment of the present invention, a hair management process for Caucasian hair types for both day time and night time applications, comprises: shampooing and conditioning the hair; drying the hair and leaving the hair damp; adding oil and moisturizing solution to the hair and scalp; covering the hair a hair cap; removing the hair cap after about 6 to about 8 hours; repeating steps nightly.

### BRIEF DESCRIPTION OF THE DRAWINGS

The structure, operation, and advantages of the present invention will become further apparent upon consideration of the following description taken in conjunction with the accompanying figures (FIGs.). The figures are intended to be illustrative, not limiting. Certain elements in some of the figures may be omitted, or illustrated not-to-scale, for illustrative clarity. The cross-sectional views may be in the form of “slices”, or “near-sighted” cross-sectional views, omitting certain background lines which would otherwise be visible in a “true” cross-sectional view, for illustrative clarity.

In the drawings accompanying the description that follows, both reference numerals and legends (labels, text descriptions) may be used to identify elements. If legends are provided, they are intended merely as an aid to the reader, and should not in any way be interpreted as limiting.

FIG. 1 is a flow chart showing the process steps for a first embodiment of a method of using a hair management process for kinky, curly hair in the natural state, in accordance with the present invention.

FIG. 2 is a flow chart showing the process steps for a second embodiment of a method of using a hair management process for heat pressed for kinky, curly hair, in accordance with the present invention.

FIG. 3 is a flow chart showing the process steps for a third embodiment of a method of using a hair management process for chemically relaxed for kinky, curly hair, in accordance with the present invention.

FIG. 4 is a flow chart showing the process steps for a fourth embodiment of a method of using a hair management process for Caucasian hair types, in accordance with the present invention.

### DETAILED DESCRIPTION OF THE PREFERRED EMBODIMENTS

In the description that follows, numerous details are set forth in order to provide a thorough understanding of the present invention. It will be appreciated by those skilled in the art that variations of these specific details are possible while still achieving the results of the present invention. Well-known processing steps are generally not described in detail in order to avoid unnecessarily obfuscating the description of the present invention.

In the description that follows, exemplary dimensions may be presented for an illustrative embodiment of the invention. The dimensions should not be interpreted as limiting. They are included to provide a sense of proportion. Generally speaking, it is the relationship between various elements, where they are located, their contrasting compositions, and sometimes their relative sizes that is of significance.

Previous and current ethnic hair solutions require harsh chemicals, damaging heat, hot iron intervention to bring hair endowment under softer manageable control, many times leading to the destruction of hair, diminished natural coloration by bleaching or total hair loss. The problem is systemic and far reaching leaving children especially vulnerable. Some other damaging standardized practices are the use of stripping relaxers using the wrong over the counter products, shampooing too often, not shampooing enough, pulling the hair too tight with styles, over processing, rough handling and split ends. The ASISTAH™ hair management process (referred to as “hair management process”) of the preferred embodiment addresses these problems with a naturopathic approach that uses the body's own systems to aid, improve, and repair itself.

The hair management process of the preferred embodiment is directed to a process which softens coarse, salon damaged, over the counter product damaged, bleached, uncared for and/or heat damaged hair. The process results in an augmented healthy hair state of deep, rich, shiny, baby soft, natural curl or wavy, regrow hair after a limited number of treatments. The hair management process uses homeostatic regulated, follicle sebaceous glandular assisted sebum lubrication, coupled with repetitive static nourishment. The nourishment is provided by an organic or inorganic matrix of natural or synthetic oils vitamins, minerals and/or fruit extracts applied to the hair and scalp daily or as needed. The hair management process also uses endothermic heat to create an eco-thermic nourishing environment.

This is achieved through intermittent and constant hair fiber, shaft, nutrient steeping. This creates a hydro-cortex populated state for follicle growth, strength, health and natural homeostatic development. The nutrient transfer uses hydrothermal steam and radiant heat to deliver hydro biometrically infused essential oils, minerals and nutrients. The infusion of the essential oils, minerals and nutrients is triggered by homeostatic, thermo regulated disambiguation intensified at the hair head and scalp by a non-permeable or permeable hair cap.

In the preferred embodiment, a non-permeable cap has been found to achieve the best results. The plastic cap raises the body's ambient temperature through its own homeostatic joules production from the head both during activity and at rest. This leads to intensified sweating of the head, particularly below the plastic cap, which hydro-biometrically diffuses from the pores in the skin and helps cleanse the skin thoroughly from impurities, such as dirt, dead skin cells, trapped chemicals and other debris. This process is repeated daily for an extended period of time, such as about seven to about twelve months, for optimum benefit and results. This period of time gives the body the needed time to re-grow a crop of new hair. The hair management process of the preferred embodiment transforms the hair through their follicle roots and shafts, once the pores and hair shafts are open by the radiant, homeostatic heat disbursement and hydro-thermic pressure from the sweating of the head. The open pores and hair shafts enable a more rapid absorption of a soluble nutrient mixture of the hydro biometrically infused essential oils, minerals and nutrients.

The use of hair mousse, a hairstyling product added to hair for extra volume and shine, in the sequencing section is a secondary illustration of physiological hair patterning by which the hair is acclimated, skin and scalp to drawing in nutrients. There is no adjutant necessitated for the functions such as combs brushes and the like.

The hair management process aids the body in repairing, restoring, and hair that suffer from salon damage, dry scalp, and unhealthy, dry, coarse, unmanageable, break away, and frizzy, tangled hair. The process creates a hydro cortex populated state for follicle growth, strength health and natural homeostatic development.

The hair management process has tertiary benefits providing the cranium, scalp and hair with conditions suitable for the production of white blood corpuscles as well as providing a boost to the body's immune process and aid for the body to fight disease causing germs and bacteria giving the body relief from congestion, colds and clearing nasal passages by loosening up mucus and allowing better brachial flow.

The hair management process helps to increase circulation and infuse nutrients, remove toxins through the skin, relax, dilate blood vessels aid the vascular system. Helps the most important organ in the body the brain, cleanse deep or surface debris and provide relief to the kidneys, while making the skin, scalp more elastic and far more flexible. The hair management process can be used to deliver liquid or powdered vitamin, minerals and drug therapy directly to the cranial receptors thereby providing access to an alternate medical treatment modality, affecting body chemistry and physiology, such as nanotechnological hydro infusion, hydro diffusion, thereto infusion, thereto diffusion, hydro biomedical infusion and thermo biomedical diffusion of compounds. The alternate medical treatment modality can affect body chemistry and physiology by introducing nutrient rich hydro biometrically infused essential oils, minerals and nutrients to the brain through the skin and the scalp.

The hair management process allows the body to adopt a natural eustress affect. This eustress affect takes a great deal of stress off the kidneys especially in the winter months when the kidneys have to work diligently to remove toxins from the body through the sweat glands which has the physiology of a nephron of the functional unit of the kidney.

The hair management process improves scalp blood flow circulation by opening hair follicles and hair shafts encouraging moisture penetration. The process gives the hair skin and scalp a greater exchange of water, essential oils, and moisture rich organic or inorganic compounds. The hair management process transforms the hair into a healthy strong, shiny soft and supple endowment. Once the hair management process opens the hair follicle, dirt, oil and product build up from styling can more easily exit the hair shaft and body. The hair management process also speeds up the body's ability to cleanse repair and enhance human pili to a natural baby soft hair endowment. The hair management process has many other benefits including: helping to moisturize and soften the hair and scalp; opening follicles and hair shafts to penetrate deep inside planting moisture where it is needed most, leaving the hair extremely soft and supple; returning the hair to its original natural coloration with body, flexibility, sheen and strength; thickening and strengthening weak hair shafts; and make the hair far more resistive to chemicals and environmental damage. A generalized example of a typical use of the hair management process consists of the following sequence of steps. First, the hair is washed with warm water daily. Each day the hair is towel dried but left damp. Then, a natural, organic or inorganic oil composition is applied to the scalp. Continuing, hair moisturizer is applied to the hair and scalp and then the hair is covered over night with a cap, such as a non-permeable plastic cap for best results. In the morning, the cap is removed and more hair moisturizer is applied liberally to the hair. Next, hair mousse is applied on top of the moisturizer, the hair is styled with fingers and the process steps of the hair management process are complete.

In general terms, there is a first stage where water hydration is used to open up pores in the scalp. The hair management process includes flooding or steeping pores and follicles through daily application of natural or synthetic oils, vitamins, fruit extract and minerals. The first stage process is particularly useful for straight hair or Caucasian hair types.

There is a second stage of the hair management process using thermic hydration. The second stage process, particularly for Afrocentric or kinky curly hair types, incorporates the use of a non permeable plastic hair cap for a minimum of once a week overnight coupled with the first stage process.

The approach of applying a moisturizer and oils daily to the hair is counter intuitive to the approach of the general hair industry, especially when It comes to straight hair or Caucasian hair types. This method helps the body repair itself at the base of the follicle. By opening the pores with water hydration, oil and moisturization, this repeditory process transforms the hair texture and body. In the case of kinky curly hair, the method shapes from a wavy curl to a spiral curl permanently. The hair management process of the present invention trains the hair and scalp to routinely absorb water, oil, vitamin and minerals mix.

The method of applying the hair management process incorporates repeditory opening of the pores in the scalp and importing nutrients, vitamins, minerals, natural and/or synthetic oils; fruit extracts and moisturization daily as an aide to the body to help it strengthen, repair, grow hair and give it elasticity and resistance to the environment.

The method of applying the hair management process incorporating using repeated water and hydro-thermic hydration routinely coupled with follicle steeping/flooding with oils and moisturization is used to repair, treat or enhance current hair endowments.

The hair management process use of plastic caps or silk caps to keep the hair body shaft and follicle in a state of hydroponic nutrient exchange is new to the present invention.

Through use of the hair cap, the pores are open making it easier for the hair follicle to receive the nutrients it needs to repair itself. The hair management process creates an environment of readily available nutrients for self repair which the body uses to enhance the shine, strength, growth and elasticity of the hair permanently.

For Caucasian hair types using the oil and moisturizer routinely is an essential step in the hair management process for hair enhancement and repair. Also, shampoo and conditioning the hair routinely is essential to the hair management process.

For kinky curly hair types using the plastic or silk cap along with a routine regimen of oil and moisturization is essential to the hair management process.

It is within the scope of the hair management process to alter any of the steps to suit the more than one hundred and eighty different hair types in duration and liquid quantity used.

The hair management process could be used in the area of nano-technology to infuse nano-information through the pores directly to the brain. That is, the hair management process could be used as a means for pharmacological delivery.

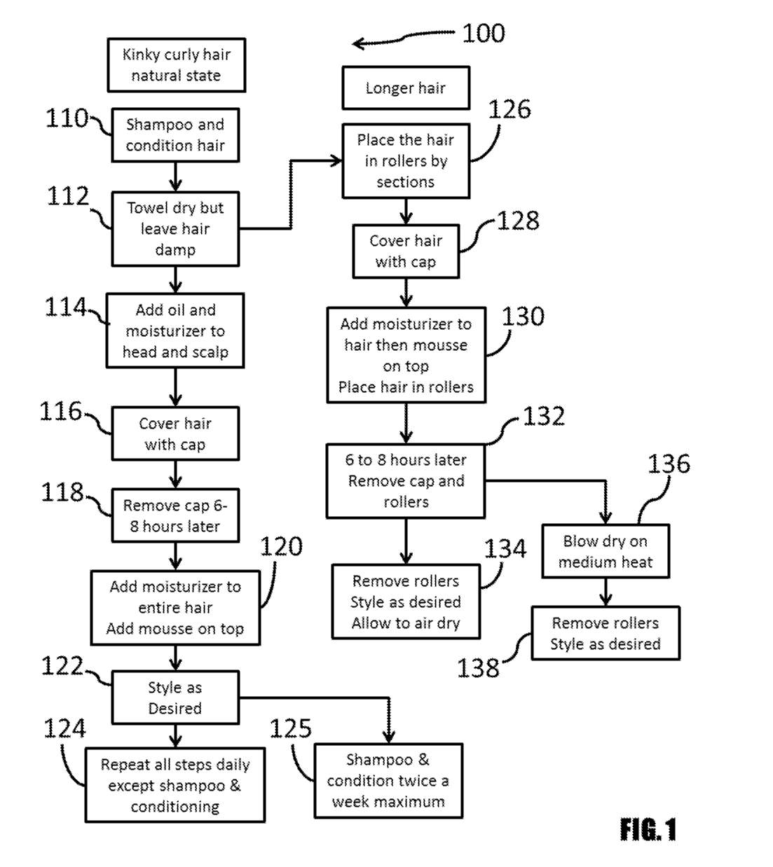

Flow chart 100 as shown in FIG. 1 shows process steps for the first embodiment of the method of using the hair management process 10 for kinky, curly hair in a natural state.

Process step 110 shows the hair first being is shampooed and conditioned. While any shampoo can be used, a preferred shampoo is a sulfate free shampoo including Water (Aqua), Disodium Laureth Sulfosuccinate, Sodium Cocoyl Isethionate, Sodium Cocoamphoacetate, Acrylates Copolymer, Glycerin, Aloe Barbadensis Leaf juice, Rosmarinus Officinalis (Rosemary) Flower Extract, Cocamidopropyl Hydroxysultaine, Cocamidopropyl Betaine, Lauryl Glucoside, Sodium Cocoyl Glutamate, Glycol Distearate, Argania Spinosa Kernel Oil, Hydrolyzed Keratin, Silicone Quaternium-16, Hydrolyzed Collagen, Trideceth-12, Quaternium-70, Propylene Glycol, Panthenol, Amodimethicone, Butyloctanol, Cetrimonium Chloride, Undeceth-11, Guar Hydroxypropyltrimonium Chloride, Undeceth-5, Chamomilla Recutita (Matricaria) Flower Extract, Petroselinum Crispum (Parsley) Extract, Citric Acid, Disodium EDTA, Caprylyl Glycol, Methylisothiazolinone, and Fragrance (Parfum). While any conditioner can be used, a preferred conditioner includes Water (Aqua), Cetearyl Alcohol, Dimethicone, Glyceryl Stearate, Cocos Nucifera (Coconut) Oil, PEG-100 Stearate, Argania Spinosa Kernel Oil, Behentrimonium Methosulfate, Cetrimonium Chloride, Stearyl Alcohol, Butyrospermum Parkii (Shea) Butter, Lanolin, Aloe Barbadensis Leaf Juice, Citric Acid, Tocopheryl Acetate (Vit E), Disodium EDTA, Glycerin, Cetrimonium Bromide, Retinyl Palmitate (Vit. A), Panthenol (Pro Vitamin B5), PEG-40 Hydrogenated Castor Oil, Glyceryl Stearate, Sorbic Acid, Potassium Sorbate, Sodium Benzoate, and Fragrance (Parfum).

As to the selection of conditioner, while any conditioner can be used, a preferred conditioner is Water (Aqua), Cetearyl Alcohol, Dimethicone, Glyceryl Stearate, Cocos Nucifera (Coconut) Oil, PEG-100 Stearate, Argania Spinosa Kernel Oil, Behentrimonium Methosulfate, Cetrimonium Chloride, Stearyl Alcohol, Butyrospermum Parkii (Shea) Butter, Lanolin, Aloe Barbadensis Leaf Juice, Citric Acid, Tocopheryl Acetate (Vit E), Disodium EDTA, Glycerin, Cetrimonium Bromide, Retinyl Palmitate (Vit. A), Panthenol (Pro Vitamin B5), PEG-40 Hydrogenated Castor Oil, Glyceryl Stearate, Sorbic Acid, Potassium Sorbate, Sodium Benzoate, and Fragrance (Parfum).

Then in process step 112, the hair is towel dried but left damp. Continuing in process step 114, oil and moisturizing solution is added to the hair and scalp. While any moisturizing solution can be used, a preferred moisturizing solution contains water (Aqua), Aloe Barbadensis Leaf Extract, Oat Beta Glucan, Glycerin, Panthenol (Pro Vitamin B5), Rosmarinus Officinalis (Rosemary) Extract, Guar Hydroxypropyltrimonium Chloride, Butyrosperium Parkii (Shea) Butter, Saccharomyces/Silicon Ferment, Cetearyl Alcohol, Behentrimonium Methosulfate, Saccharomyces/Magnesium Ferment, Saccharomyces/Iron Ferment, Saccharomyces/Zinc Ferment, Tocophery Acetate (Vit E), Retinyl Palmitate (Vit A), Saccharomyces/Copper Ferment, Allium Sativum (Garlic) Bulb Extract, Dimethicone, Caprylyl Glycol, Dosodium EDTA, Methylisothiazolinone, Potassium Sorbate, and Fragrance (Parfum).

While any oil can be used, a preferred oil contains Cyclopentasiloxane, Dimethicone/Vinyl Dimethicone Crosspolymer Argania Spinosa Kernel Oil, Dimethiconol, Cocos Nucifera Coconut Oil, Chamomilla Recutita (Matricaria) Flower Extract, Caprylic/Capric Triglyceride, Tocopheryl Acetate, Retinyl Palmitate, and Fragrance (Parfum).

Continuing in process step 116, the hair is covered with a non-permeable or permeable hair cap. Then in process step 118, the hair cap is removed from the hair after about 6 to about 8 hours.

Continuing in process step 120, moisturizer is added to the hair and then hair mousse is added to the top of the hair. While any hair mousse can be used, a preferred hair mousse contains water (Aqua), PVP, Oat Beta Glucan, Pyrus Malus (Apple) Fruit Extract, Vaccinium Angustifolium (Blueberry) Fruit Extract, Polyimide-1, Aloe Barbadensis Leaf Juice, Hydrolyzed Wheat Protein, Hydrolyzed Collagen, Chamomilla Recruita (Matricaria) Flower Extract, Cocamidopyl Betaine, Symphytum Officinale Leaf Extract, Echinacea Purpurea Extract, Allium Sativum (Garlic) Bulb Extract, Citrus Limon (Lemon) Flower/Leaf/Stem Extract, Salvia Officinallis (Sage) Flower/Leaf/Stem Extract, Viola Tricolor Extract, Trigonella Foenum-Graecum Seed Extract, Citrus Limon (Lemon) Fruit Extract, Propolis Extract, Spurilina Platensis Extract, Quassia Amara Wood Extract, Caprylyl Glycol, Methylisothiazolinone, Disodium EDTA and Fragrance (Parfum).

Continuing in process step 122, the hair is styled as desired. In process step 124, all of the steps 110 through 122 are repeated on a daily basis except the shampooing and conditioning are done a maximum of twice a week as in process step 125.

Referring again to the flow chart 100 as shown in FIG. 1, there are shown additional process steps for the first embodiment of the method of using the hair management process 10 for kinky, curly hair in a natural state where the hair is 2 or more inches long.

As previously discussed, process step 110 shows the hair first being is shampooed and conditioned. While any shampoo can be used, a preferred shampoo is a sulfate free shampoo as previously described.

Then in step 112, the hair is towel dried but left damp. Continuing in process step block 114, oil and moisturizing solution is added to the hair and scalp. While any oil and moisturizing solution can be used, a preferred oil and moisturizing solution is one previously described.

Then in process step 126, the hair is placed in rollers by sections.

Continuing in process step 128, the hair is covered with a non-permeable or permeable hair cap. When in process step 130, the rollers and the hair cap are removed from the hair after about 6 to about 8 hours.

Continuing in process step 132, moisturizer is added to the hair and then hair mousse is added to the top of the hair. While any hair mousse can be used, a preferred hair mousse is described herein before. Then the hair is again placed in rollers.

Continuing in process step 134, the rollers are removed and the hair is styled as desired. Then the hair is allowed to air dry.

Alternatively, after process step 136, the hair is blown dry on medium heat. Finally in process step 138, the rollers are removed and the hair is styled as desired.

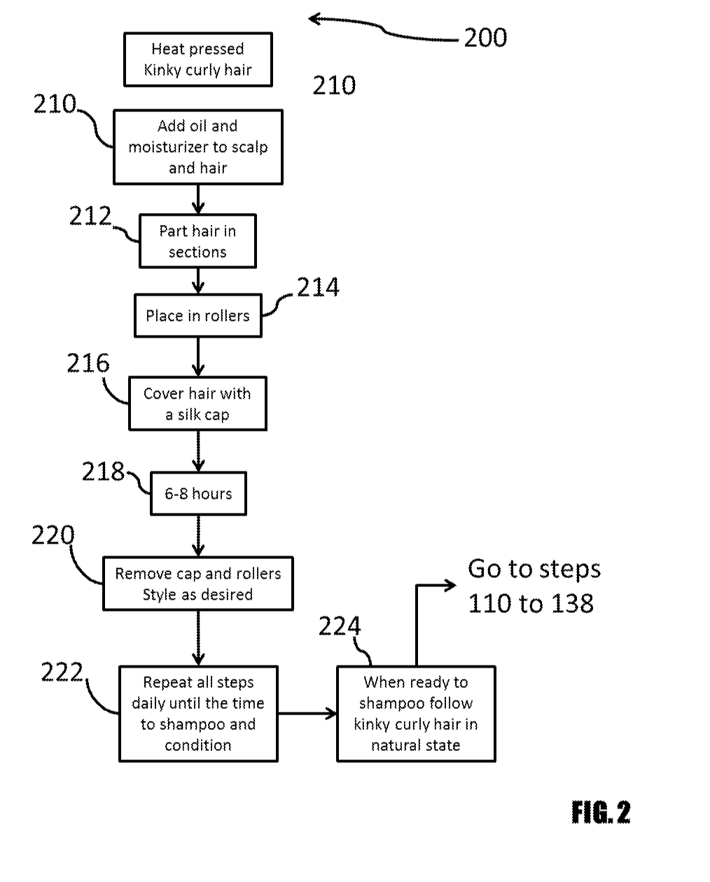

Referring again to the flow chart 200 as shown in FIG. 2, there are shown process steps for an embodiment of the method of using the hair management process 200 for heat pressed, kinky, curly hair.

In process step block 210, oil and moisturizing solution is added to the scalp and hair. While any oil and moisturizing solution can be used, a preferred oil and moisturizing solution is one previously described.

Then in process step 212, the hair is parted in sections and then in process step 214 placed in rollers.

Continuing in process step 216, the hair is covered with a non-permeable or permeable hair cap. Then in process step 218, wait a period of about 6 to about 8 hours. Then in process step 220, the rollers and the hair cap are removed from the hair and the hair is styled as desired.

Continuing, process steps 210 through 220 are repeated until one desires to shampoo and condition the hair. Then, when the hair is to be shampooed, steps process steps 110 through 138 are followed.

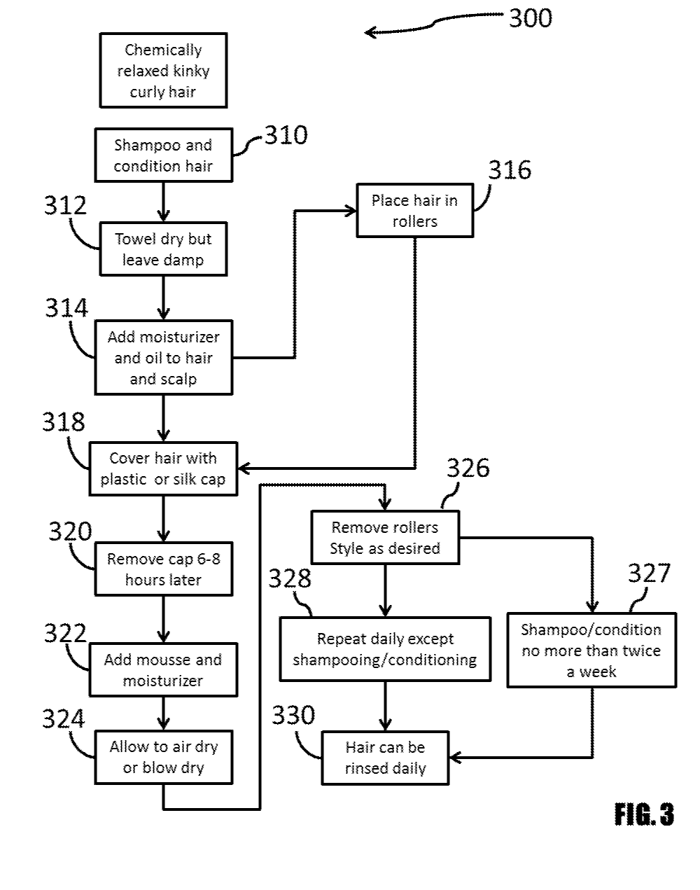

Referring again to the flow chart 300 as shown in FIG. 3, there are shown additional process steps for the first embodiment of the method of using the hair management process 300 for chemically relaxed, kinky, curly hair.

As previously discussed, process step 310 shows the hair first being is shampooed and conditioned. While any shampoo can be used, a preferred shampoo is a sulfate free shampoo as previously described.

Then in step 312, the hair is towel dried but left damp. Continuing in process step 314, oil and moisturizing solution is added to the hair and scalp. While any oil and moisturizing solution can be used, a preferred oil and moisturizing solution is one previously described.

Then, if desired, in process step 316, the hair is placed in rollers by sections. Alternatively, after adding the moisturizer and oil to the hair and scalp, the hair is covered with a non-permeable or permeable hair cap made of plastic or silk in process step 318. Then in process step 320, the hair cap is removed from the hair after about 6 to about 8 hours.

Continuing in process step 322, moisturizer is added to the hair and then hair mousse is added to the top of the hair. While any hair mousse can be used, a preferred hair mousse is described herein before.

Continuing in process step 324, the hair is blown thy or allowed to air dry.

Alternatively, after process step 326, the rollers are removed and the hair is styled as desired.

Continuing in process step 328, process steps 310 through 326 are repeated on a daily basis except the shampooing and conditioning are done a maximum of twice a week as in process step 327. Continuing in process step 330, the hair can be rinsed daily.

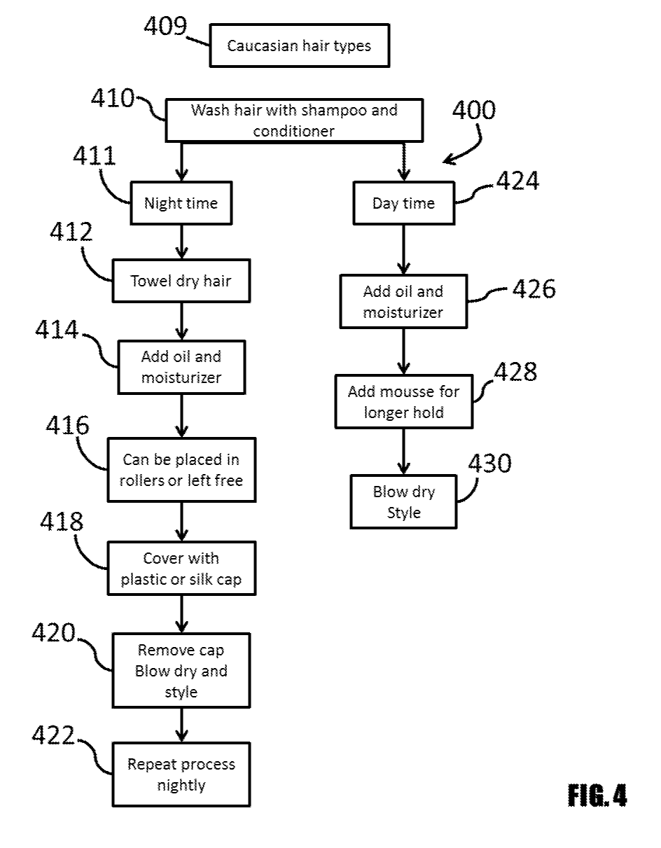

Referring to the flow chart 400 as shown in FIG. 4, there are process steps for an embodiment of the method of using the hair management process 409 for Caucasian hair types for both day time and night time applications.

Process step 410 shows the hair first being is shampooed and conditioned. While any shampoo can be used, a preferred shampoo is a sulfate free shampoo as previously described.

Then in step 411, the night time operation begins. First, in process step 412, hair is towel dried. Continuing in process step 414, oil and moisturizing solution is added to the hair and scalp. While any oil and moisturizing solution can be used, a preferred oil and moisturizing solution is one previously described.

Then, if desired, in process step 416, the hair can be placed in rollers or left free. Then in process step 418, the hair is covered with a non-permeable or permeable hair cap made of plastic or silk.

Then in process step 420, the hair cap is removed from the hair, typically after a night of sleep, and the hair is blown dry and styled. This process, as indicated in process step 422 is repeated nightly.

Alternatively, in step 424, the day time operation begins. Note that the daytime operation can be employed with or without the night time operation. First, in process step 426, oil and moisturizing solution is added to the hair and scalp. While any oil and moisturizing solution can be used, a preferred oil and moisturizing solution is one previously described.

Then, if desired, in process step 428, hair mousse is added to the hair for longer hold. While any hair mousse can be used, a preferred hair mousse is described herein before.

Continuing in process step 430, the hair is blown dry and styled.

Although the invention has been shown and described with respect to a certain preferred embodiment or embodiments, certain equivalent alterations and modifications will occur to others skilled in the art upon the reading and understanding of this specification and the annexed drawings. In particular regard to the various functions performed by the above described elements and components (assemblies, devices, etc.) the terms (including a reference to a “means”) used to describe such elements and components are intended to correspond, unless otherwise indicated, to any element and/or component which performs the specified function of the described element and/or component (i.e., that is functionally equivalent), even though not structurally equivalent to the disclosed process and/or structure which performs the function in the herein illustrated exemplary embodiments of the invention. In addition, while a particular feature of the invention may have been disclosed with respect to only one of several embodiments, such feature may be combined with one or more features of the other embodiments as may be desired and advantageous for any given or particular application.

1. A hair management process comprising the steps of:
washing the hair and scalp;
towel drying the hair and leaving the hair damp;
applying an oil composition to the scalp;
applying hair deep moisturizer to the hair and scalp;
covering the hair with a cap either permeable or non permeable;
minimum sequence of two times weekly to daily
creating a repeatable endo-thermic environment for hair growth
removing the cap and applying hair moisturizer to the hair;
applying hair mousse on top of the moisturizer; and
styling the hair.

2. The hair management process of claim 1 including covering the hair with a permeable or non permeable cap for a period of about 6 to about 8 hours.

3. The hair management process of claim 2 including causing water hydration within the non permeable cap to open pores in the scalp whereby the pores and hair follicles are flooded and steeped with the oil composition. Creating an endo-thermic environment for hair growth

4. The hair management process of claim 3 including selecting the oil composition from a group consisting of natural oils, synthetic oils, vitamins, fruit extract and minerals.

5. The hair management process of claim 2 including covering the hair with a permeable or non permeable cap for a period of about 6 to about 8 hours for at least twice a week.

6. The hair management process of claim 5 including covering the hair with a permeable or non permeable cap for a period of about 6 to about 8 hours for at least once a week.

7. A hair management process for kinky, curly hair comprising the steps of:
shampooing and conditioning the hair in a natural state;
partial drying the hair and leaving the hair damp;
adding oil and moisturizing solution to the hair and scalp;
covering the hair with a permeable or non-permeable cap;
removing the hair cap after about 6 to about 8 hours;
moisturizing the hair and adding hair mousse to the top of the hair; and
styling the hair.

8. The hair management process of claim 7 including shampooing and conditioning the hair a maximum of twice a week.

9. The hair management process of claim 7 If the hair is long enough further including placing the hair in rollers by sections after towel drying the hair.

10. The hair management process of claim 7 including covering the hair with a permeable cap after adding oil and moisturizing solution to the hair and scalp.

11. The hair management process of claim 10 including:
placing the hair in rollers after for deep moisturizing the hair and adding hair mousse to the top of the hair; and
removing the rollers 6 to 8 hours later and styling the hair to desired look.

12. The hair management process of claim 11 including removing the hair cap and rollers after about 6 to about 8 hours.

13. The hair management process of claim 12 including:
blow drying the hair before removing the rollers and styling the hair.

14. The hair management process of claim 7 for heat pressed kinky curly hair including:
adding oil and deep moisturizing solution at least three times weekly to the scalp and hair after shampooing and conditioning the hair no more than once weekly;
parting the hair in sections and placing rollers in the hair;
covering the hair with a hair cap;
after a period of about 6 to about 8 hours remove the rollers and hair cap and style the hair;
repeat steps until hair ready to shampoo and condition again.

15. A hair management process for chemically relaxed curly hair comprising the steps of:
shampooing and conditioning the hair;
drying the hair and leaving the hair damp;
adding oil and moisturizing solution to the hair and scalp;
covering the with a permeable or non-permeable hair cap;
removing the hair cap after about 6 to about 8 hours;
moisturizing the hair and adding hair mousse to the top of the hair;
blow drying the hair on low heat setting;
repeating steps daily except shampooing and conditioning; and
rinsing hair daily.

16. The hair management process of claim 15 including;
placing hair in rollers after adding oil and moisturizing solution to the hair and scalp; and
removing rollers and style hair after drying the hair.

17. The hair management process of claim 16 including;
shampooing and conditioning hair no more than twice a week after removing rollers and
styling the hair.

18. A hair management process for Caucasian hair types for day time and or night time applications, not both three times weekly comprising the steps of:
shampooing and conditioning the hair;
drying the hair and leaving the hair damp;
adding oil and moisturizing solution to the hair and scalp;
covering the hair with a permeable or non-permeable cap;
removing the hair cap after about 6 to about 8 hours;
repeating steps nightly.

19. The hair management process of claim 18 including;
shampooing and conditioning the hair during the day and or night time not both.

20. The hair management process of claim 18 including;
shampooing and conditioning the hair during the day and or night time not both;
adding oil and deep moisturizing solution to the hair and scalp before drying the hair;
adding hair mousse to the hair; and blow dry the hair and style to desired look.

21. The hair management process of claim 20 including;
Reduces the need to wash Caucasian hair daily

22. The hair management process of claim 21 including;
resolves the problem of offensive smell due to lack of washing, with oil and deep moisturizing solution application frequency.
